(19) United States
(12) Patent Application Publication
(10) Pub. No.: US 2009/0247851 A1
(43) Pub. Date: Oct. 1, 2009

(54) Graphical User Interface For Monitor Alarm Management

(75) Inventors: Keitch Batchelder, New York, NY (US); Scott Amundson, Oakland, CA (US); Steve Vargas, Sun Valley, CA (US); James Ochs, Seattle, WA (US); Li Li, Milpitas, CA (US); Robin Boyce, Pleasanton, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/409,710

(22) Filed: Mar. 24, 2009

Related U.S. Application Data
(60) Provisional application No. 61/070,838, filed on Mar. 26, 2008.

(51) Int. Cl. G06F 3/048; A61B 5/1455

(52) U.S. Cl. 600/324; 715/772

(57) ABSTRACT

The present disclosure provides a system and method for facilitating user input of alarm settings for a patient monitor. In various embodiments, a pulse oximetry monitor may include a graphical user interface (GUI) which is capable of displaying a graph of blood oxygen saturation percentage over time. The system may be capable of allowing a user to enter an alarm threshold value and/or an alarm integration threshold value. The alarm threshold value may be displayed as a line on the graph, and the alarm integration threshold value may be displayed as a shaded area on the graph. The GUI may include an indicator of where an alarm would be initiated given the graph, the input alarm threshold value, and/or the alarm integration threshold value. The disclosed GUI may provide the user with a clear illustration of how the alarm threshold value and alarm integration threshold value may affect the alarm.

### RELATED APPLICATIONS

This application claims priority to U.S. Provisional Application No. 61/070,838, filed Mar. 26, 2008, and is incorporated herein by reference in its entirety.

### BACKGROUND

The present disclosure relates to a user interface for alarm monitor management.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of healthcare, caregivers (e.g., doctors and other healthcare professionals) often desire to monitor certain physiological characteristics of their patients. Accordingly, a wide variety of monitoring devices have been developed for monitoring many such physiological characteristics. These monitoring devices often provide doctors and other healthcare personnel with information that facilitates provision of the best possible healthcare for their patients. As a result, such monitoring devices have become a perennial feature of modern medicine.

One technique for monitoring physiological characteristics of a patient is commonly referred to as pulse oximetry, and the devices built based upon pulse oximetry techniques are commonly referred to as pulse oximeters. Pulse oximeters may be used to measure and monitor various blood flow characteristics of a patient. For example, a pulse oximeter may be utilized to monitor the blood oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. A photo-plethysmographic waveform, which corresponds to the cyclic attenuation of optical energy through the patient's tissue, may be generated from the detected light. Additionally, one or more of the above physiological characteristics may be calculated based upon the amount of light absorbed or scattered. More specifically, the light passed through the tissue may be selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

In addition to monitoring a patient's physiological characteristics, a pulse oximeter or other patient monitor may alert a caregiver when certain physiological conditions are recognized. For example, a normal range for a particular physiological parameter of a patient may be defined by setting low and/or high threshold values for the physiological parameter, and an alarm may be generated by the monitor when a detected value of the physiological parameter is outside the normal range. When activated, the alarm may alert the caregiver to a problem associated with the physiological parameter being outside of the normal range. The alert may include, for example, an audible and/or visible alarm on the oximeter or an audible and/or visible alarm at a remote location, such as a nurse station. These patient monitors may generally be provided with default alarm thresholds. However, in some instances, it may be desirable to alter the thresholds for various reasons.

### BRIEF DESCRIPTION OF THE DRAWINGS

Advantages of the disclosure may become apparent upon reading the following detailed description and upon reference to the drawings in which:

FIG. 1 is a graph illustrating a patient's measured SpO₂ versus time in accordance with embodiments;

FIG. 2 is a perspective view of a pulse oximeter coupled to a multi-parameter patient monitor and a sensor in accordance with embodiments;

FIG. 3 is a block diagram of the pulse oximeter and sensor coupled to a patient in accordance with embodiments; and

FIGS. 4-8 are exemplary graphical user interfaces of the pulse oximeter in accordance with embodiments.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Different patients may exhibit different normal ranges of physiological characteristic values. Factors such as age, weight, height diagnosis, and a patient's use of certain medications may affect the patient's normal ranges of physiological parameters. For example, with a neonate, the normal SpO₂ range may be 80-95 percent. In contrast, for a 40-year-old patient, the normal SpO₂ range may be 85-100 percent. Accordingly, it may be desirable to set different low and/or high thresholds for particular parameters based on the patient being monitored.

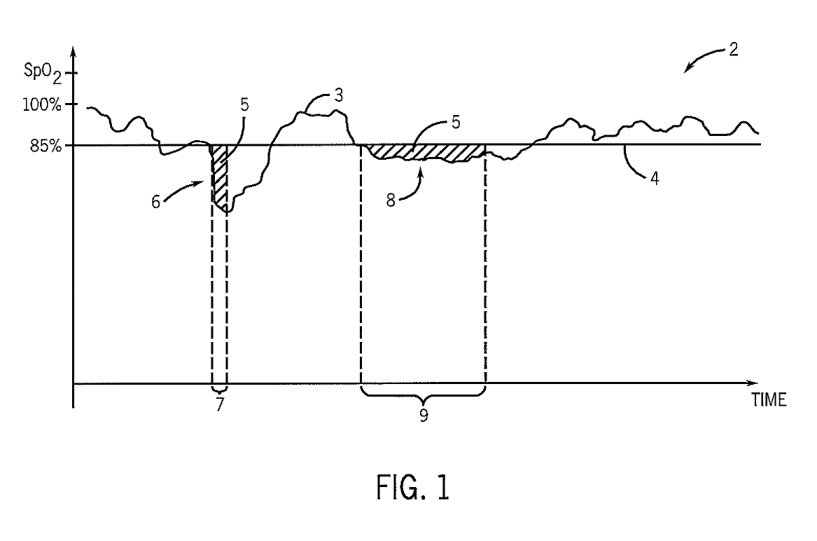

In addition, simply monitoring a patient's physiological parameters may result in excessive alarms if a parameter repeatedly exceeds a threshold only momentarily. Accordingly, an alarm integration method may be employed to reduce nuisance alarms on patient monitors. An exemplary alarm management system may be the SatSeconds™ alarm management technology available, for example, in the OxiMax® N-600x™ pulse oximeter available from Nellcor Puritan Bennett, LLC, or Covidien. Generally speaking, SatSeconds alarm management operates by integrating an area between an alarm threshold and a patient's measured physiological parameters over time. For example, a patient's SpO₂ readings may be charted, as in a graph 2 illustrated in FIG. 1. The patient's SpO₂ readings may be displayed as a plot 3 in the graph 2. Similarly, a threshold SpO₂ value (e.g., 85 or 90 percent) may be displayed as a line 4 in the graph 2. Rather than sounding an alarm as soon as the patient's measured SpO₂ (plot 3) drops below the threshold value (line 4), the SatSeconds system measures an area 5 (shaded in FIG. 1) by integrating the difference between the plot 3 and the line 4 when the plot 3 is below the line 4. The area 5 may be known as the SatSeconds value because it is a measure of saturation versus time. When the SatSeconds value exceeds a threshold value (e.g., a preset threshold or a user-input threshold), the caregiver may be alerted that the patient's oxygen saturation is too low. Due to the nature of this technology, a significant desaturation event 6 (e.g., a large drop in SpO₂) may cause the alarm to activate quickly because the SatSeconds threshold value may be exceeded in a short period of time 7. In contrast, a minor desaturation event 8 (e.g., a drop in SpO₂ (line 4) to just below the threshold (line 6)) may not cause the alarm to be activated quickly. That is, the minor desaturation event 8 may continue for a relatively long period of time 9 before the SatSeconds threshold value is exceeded. Exemplary SatSeconds threshold values may range from 0-200, where a threshold of 0 SatSeconds results in the alarm being activated as soon as the patient's measured SpO₂ (plot 3) drops below the threshold value (line 4).

Because the SatSeconds technology is relatively new in the medical field, it may be desirable to assist the caregiver in efficiently determining the desired SatSeconds threshold value. Accordingly, a patient monitoring system in accordance with embodiments of the present disclosure may include one or more user interfaces which enable the caregiver to change the SatSeconds threshold value and/or the SpO₂ threshold value. In addition, the user interfaces may include graphical representations, as described below, to assist the caregiver in determining the optimal thresholds for a patient. Although the techniques introduced above and discussed in detail below may be implemented for a variety of medical devices, the present disclosure will discuss the implementation of these techniques in a pulse oximetry system.

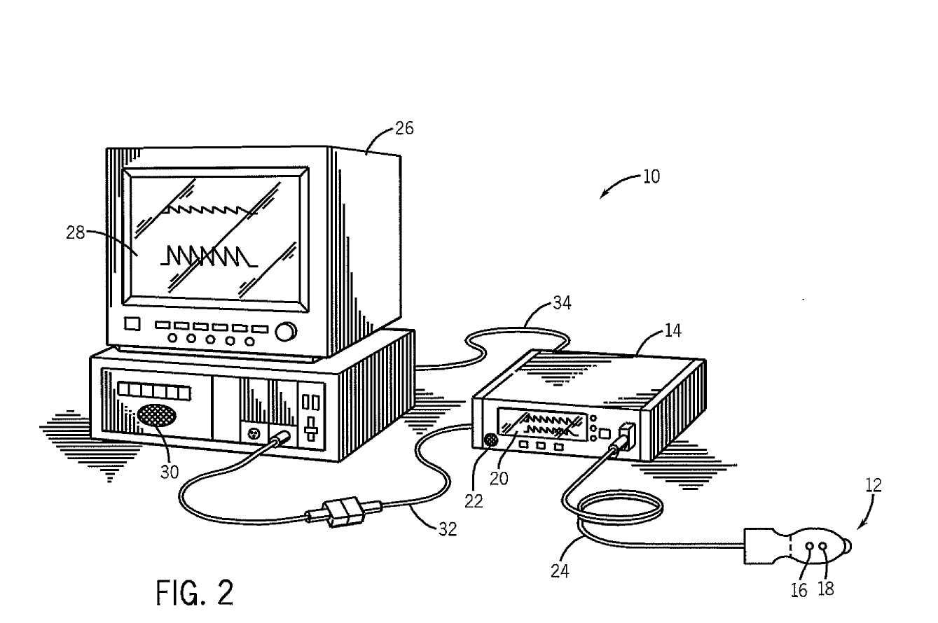

FIG. 2 is a perspective view of such a pulse oximetry system 10 in accordance with an embodiment. The system 10 includes a sensor 12 and a pulse oximetry monitor 14. The sensor 12 includes an emitter 16 for emitting light at certain wavelengths into a patient's tissue and a detector 18 for detecting the light after it is reflected and/or absorbed by the patient's tissue. The monitor 14 may be configured to calculate physiological parameters received from the sensor 12 relating to light emission and detection. Further, the monitor 14 includes a display 20 configured to display the physiological parameters, other information about the system, and/or alarm indications. The monitor 14 also includes a speaker 22 to provide an audible alarm in the event that the patient's physiological parameters exceed a threshold. The sensor 12 is communicatively coupled to the monitor 14 via a cable 24. However, in other embodiments a wireless transmission device (not shown) or the like may be utilized instead of or in addition to the cable 24.

In the illustrated embodiment, the pulse oximetry system 10 also includes a multi-parameter patient monitor 26. In addition to the monitor 14, or alternatively, the multi-parameter patient monitor 26 may be configured to calculate physiological parameters and to provide a central display 28 for information from the monitor 14 and from other medical monitoring devices or systems (not shown). For example, the multi-parameter patient monitor 26 may be configured to display a patient's SpO₂ and pulse rate information from the monitor 14 and blood pressure from a blood pressure monitor (not shown) on the display 28. Additionally, the multi-parameter patient monitor 26 may emit a visible or audible alarm via the display 28 or a speaker 30, respectively, if the patient's physiological parameters are found to be outside of the normal range. The monitor 14 may be communicatively coupled to the multi-parameter patient monitor 26 via a cable 32 or 34 coupled to a sensor input port or a digital communications port, respectively. In addition, the monitor 14 and/or the multi-parameter patient monitor 26 may be connected to a network to enable the sharing of information with servers or other workstations (not shown).

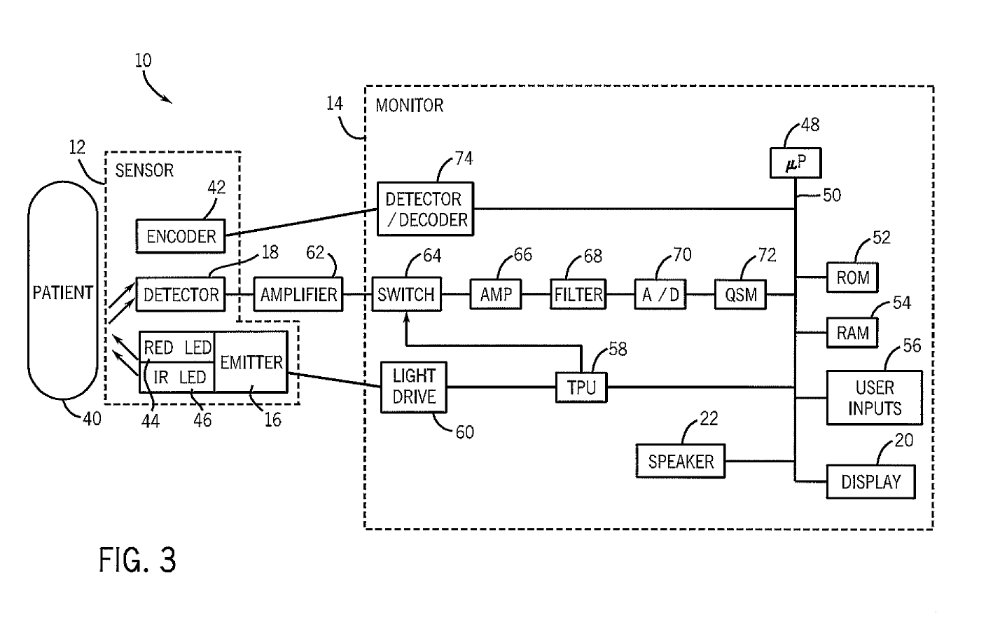

FIG. 3 is a block diagram of the exemplary pulse oximetry system 10 of FIG. 1 coupled to a patient 40 in accordance with present embodiments. One such pulse oximeter that may be used in the implementation of the present technique is the OxiMax® N-600x™ available from Nellcor Puritan Bennett LLC, but the following discussion may be applied to other pulse oximeters and medical devices. Specifically, certain components of the sensor 12 and the monitor 14 are illustrated in FIG. 2. The sensor 12 may include the emitter 16, the detector 18, and an encoder 42. It should be noted that the emitter 16 may be configured to emit at least two wavelengths of light, e.g., RED and IR, into a patient's tissue 40. Hence, the emitter 16 may include a RED LED 44 and an IR LED 46 for emitting light into the patient's tissue 40 at the wavelengths used to calculate the patient's physiological parameters. In certain embodiments, the RED wavelength may be between about 600 nm and about 700 nm, and the IR wavelength may be between about 800 nm and about 1000 nm. Alternative light sources may be used in other embodiments. For example, a single wide-spectrum light source may be used, and the detector 18 may be configured to detect light only at certain wavelengths. In another example, the detector 18 may detect a wide spectrum of wavelengths of light, and the monitor 14 may process only those wavelengths which are of interest. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present techniques.

In one embodiment, the detector 18 may be configured to detect the intensity of light at the RED and IR wavelengths. In operation, light enters the detector 18 after passing through the patient's tissue 40. The detector 18 may convert the intensity of the received light into an electrical signal. The light intensity may be directly related to the absorbance and/or reflectance of light in the tissue 40. That is, when more light at a certain wavelength is absorbed or reflected, less light of that wavelength is typically received from the tissue by the detector 18. After converting the received light to an electrical signal, the detector 18 may send the signal to the monitor 14, where physiological parameters may be calculated based on the absorption of the RED and IR wavelengths in the patient's tissue 40.

The encoder 42 may contain information about the sensor 12, such as what type of sensor it is (e.g., whether the sensor is intended for placement on a forehead or digit) and the wavelengths of light emitted by the emitter 16. This information may allow the monitor 14 to select appropriate algorithms and/or calibration coefficients for calculating the patient's physiological parameters. The encoder 42 may, for instance, be a coded resistor which stores values corresponding to the type of the sensor 12 and/or the wavelengths of light emitted by the emitter 16. These coded values may be communicated to the monitor 14, which determines how to calculate the patient's physiological parameters. In another embodiment the encoder 42 may be a memory on which one or more of the following information may be stored for communication to the monitor 14: the type of the sensor 12; the wavelengths of light emitted by the emitter 16; and the proper calibration coefficients and/or algorithms to be used for calculating the patient's physiological parameters. Exemplary pulse oximetry sensors configured to cooperate with pulse oximetry monitors are the OxiMax® sensors available from Nellcor Puritan Bennett LLC.

Signals from the detector 18 and the encoder 42 may be transmitted to the monitor 14. The monitor 14 generally may include processors 48 connected to an internal bus 50. Also connected to the bus may be a read-only memory (ROM) 52, a random access memory (RAM) 54, user inputs 56, the display 20, or the speaker 22. A time processing unit (TPU) 58 may provide timing control signals to a light drive circuitry 60 which controls when the emitter 16 is illuminated and the multiplexed timing for the RED LED 44 and the IR LED 46. The TPU 58 control the gating-in of signals from detector 18 through an amplifier 62 and a switching circuit 64. These signals may be sampled at the proper time, depending upon which light source is illuminated. The received signal from the detector 18 may be passed through an amplifier 66, a low pass filter 68, and an analog-to-digital converter 70. The digital data may then be stored in a queued serial module (QSM) 72 for later downloading to the RAM 54 as the QSM 72 fills up. In one embodiment, there may be multiple separate parallel paths having the amplifier 66, the filter 68, and the A/D converter 70 for multiple light wavelengths or spectra received.

The processor(s) 48 may determine the patient's physiological parameters, such as SpO₂ and pulse rate, using various algorithms and/or look-up tables based on the value of the received signals corresponding to the light received by the detector 18. Signals corresponding to information about the sensor 12 may be transmitted from the encoder 42 to a decoder 74. The decoder 74 may translate these signals to enable the microprocessor to determine the proper method for calculating the patient's physiological parameters, for example, based on algorithms or look-up tables stored in the ROM 52. In addition, or alternatively, the encoder 42 may contain the algorithms or look-up tables for calculating the patient's physiological parameters. The user inputs 56 may be used to change alarm thresholds for measured physiological parameters on the monitor 14, as described below. In certain embodiments, the display 20 may exhibit a minimum SpO₂ threshold and a selection of SatSeconds values, which the user may change using the user inputs 56. The monitor 14 may then provide an alarm when the patient's calculated SpO₂ integral exceeds the SatSeconds threshold.

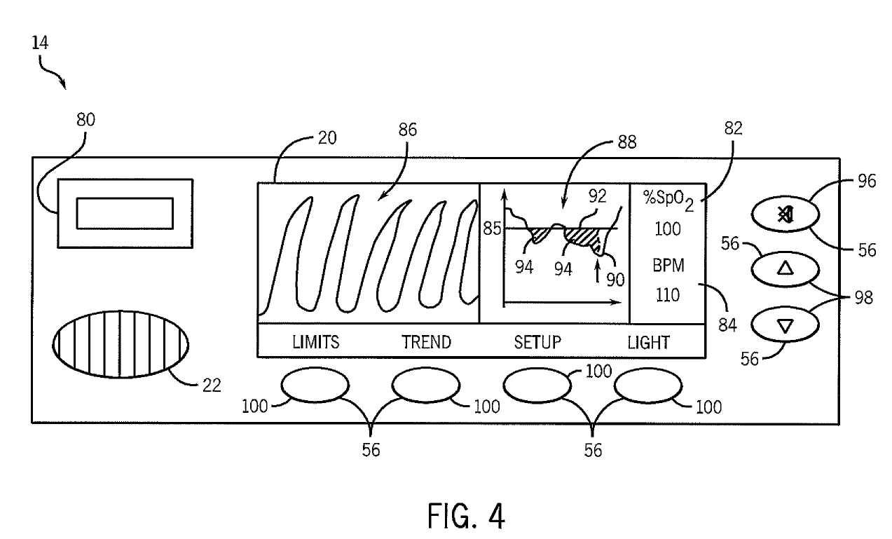

FIG. 4 illustrates an exemplary monitor 14 for use in the system 10 (FIG. 2). The monitor 14 may generally include the display 20, the speaker 22, the user inputs 56, and a communication port 80 for coupling the sensor 12 (FIG. 2) to the monitor 14. The display 20 may generally show an SpO₂ value 82 (i.e., percentage), a pulse rate 84 (i.e., beats per minute), a plethysmographic waveform (i.e., a plot 86), and a graphical representation 88 of the measured SpO₂ value versus time (i.e., a plot 90). In addition to displaying a trend of the patient's SpO₂ value, the graph 88 may serve as an indicator of the SatSeconds value. For example, a set SpO₂ threshold value (i.e., a line 92) may be displayed on the graph 88 with the plot 90. When the measured SpO₂ value (i.e., the plot 90) drops below the threshold value (i.e., the line 92), an area 94 between the plot 90 and the line 92 may begin to fill in on the display 14. At this time, the monitor 14 may begin to integrate the difference between the measured SpO₂ value (i.e., the plot 90) and the threshold value (i.e., the line 92). When the area 94 reaches a set value (i.e., the SatSeconds threshold value), the monitor 14 may indicate to the caregiver that a desaturation event is occurring, for example, by sounding an alarm via the speaker 22, displaying an alert message on the display 20, sending a signal to a nurse's station, or otherwise providing a notification that the patient's physiological parameters are not normal.

The user inputs 56 may enable the caregiver to control the monitor 14 and change settings, such as the SpO₂ threshold value and/or the SatSeconds threshold value. For example, an alarm silence button 96 may enable the caregiver to silence an audible alarm (e.g., when the patient is being cared for), and volume buttons 98 may enable the caregiver to adjust the volume of the alarm and/or any other indicators emitted from the speaker 22. In addition, soft keys 100 may correspond to variable functions, as displayed on the display 22. The soft keys 100 may provide access to further data displays and/or setting displays, as described further below. Soft keys 100 provided on the display 20 may enable the caregiver to see and/or change alarm thresholds, view different trend data, change characteristics of the display 20, turn a backlight on or off, or perform other functions.

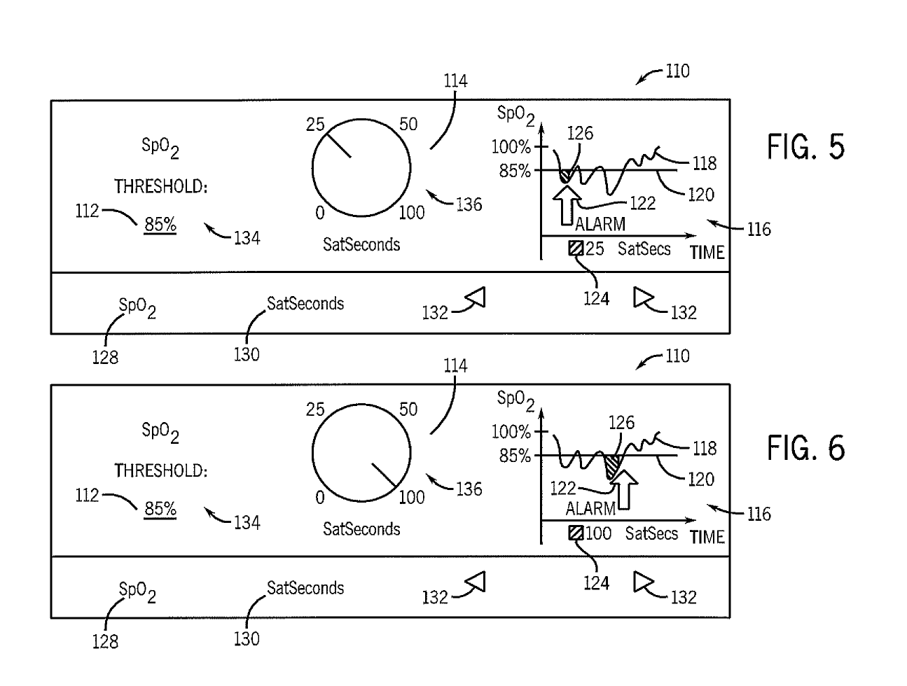

As indicated, the caregiver may access an alarm threshold control display 110, an embodiment of which is illustrated in FIG. 5, by selecting the limits soft key 100 (FIG. 4). The alarm threshold control display 110 may enable the caregiver to view and/or change both an SpO₂ threshold 112 and a SatSeconds threshold 114. In addition, a graphical representation 116 of the effect of the SpO₂ threshold 112 and the SatSeconds threshold 114 may be provided. The graphical representation 116 may include, for example, an exemplary SpO₂ plot 118 and a line 120 corresponding to the SpO₂ threshold 112. As will be illustrated further, the exemplary SpO₂ plot 118 may remain constant so that the caregiver can clearly see how changes to the SpO₂ threshold 112 and the SatSeconds threshold 114 will affect the alarm settings.

Based on the SpO₂ threshold 112 and the SatSeconds threshold 114, an alarm indicator 122 may illustrate the time at which the alarm would be sounded in the SpO₂ plot 118. That is, given the SpO₂ plot 118 and the thresholds 112 and 114, the monitor 14 (FIG. 2) would alert the caregiver to a problem at the point indicated by the alarm indicator 122. A shaded symbol 124 may correspond to the SatSeconds threshold 114 to indicate to the caregiver the size of an area 126 between the threshold line 120 and the plot 118 which must be filled before the alarm would go off. Furthermore, the first area 126 which corresponds to the SatSeconds threshold 114 may be shaded in to enable the caregiver to see where the SatSeconds threshold 114 is first exceeded on the exemplary SpO₂ plot 118. The shaded in area 126 may correspond to the alarm indicator 122.

The thresholds 112 and 114 may be changed via soft keys. For example, an SpO₂ soft key 128 may be selected to change the SpO₂ threshold 112, or a SatSeconds soft key 130 may be selected to change the SatSeconds threshold 114. Selection of the threshold 112 or 114 may be indicated, for example, by a backlight, a color change, an underline, or any other indication method. The threshold 112 or 114 may then be changed by pressing increment soft keys 132. The left increment soft key 132 may be pressed to decrease the threshold 112 or 114, while the right increment soft key 132 increases the threshold 112 or 114. It should be understood that the position of the increment soft keys 132 may be reversed. The increment soft keys 132 may be up and down arrows, left and right arrows, a minis sign and a plus sign, “UP” and “DOWN,” or any other indicator which enables the caregiver to clearly adjust the thresholds 112 and 114. The thresholds 112 and 114 may be displayed as a numerical value 134 (e.g., the SpO₂ threshold 112), a virtual knob 136 (e.g., the SatSeconds threshold), or any other value indicator. In addition, the thresholds 112 and 114 may be adjusted in increments of any size. For example, the SpO₂ threshold 112 may be adjusted in increments of 1% while the SatSeconds threshold 114 may be adjusted in increments of 25. A number of discreet values may be available for the thresholds 112 and 114, or the value adjustment may be continuous.

As described above, changes in the thresholds 112 and/or 114 are illustrated in the graphical representation 116. While the SpO₂ plot 118 remains constant, the threshold line 120 may move up or down based on changes to the SpO₂ threshold. Furthermore, in the case of a color display 110, the SpO₂ threshold value 112 and the line 120 may be the same color, which is different from the other colors in the graphical representation 116. Similarly, the SatSeconds symbol 124 and the area 126 may change based on the SatSeconds threshold 114. The SatSeconds threshold 114, symbol 124, and area 126 may be illustrated in the same color, which is different from the other colors on the display 110. By color-coding the display 110, the caregiver may further see how the threshold values 112 and 114 affect the alarm settings. In addition, the SatSeconds symbol 124 may take on various forms to further illustrate the differences in SatSeconds thresholds 114. For example, the symbol 124 may be a square which varies in size based on the threshold 114, or the symbol 124 may be a square of constant size which fills up based on the threshold 114.

FIGS. 5-7 illustrate how changes in the SpO₂ threshold 112 and the SatSeconds threshold 114 are illustrated in the graphical representation 116. For example, in FIG. 5 the SatSeconds threshold 114 is increased from 25 (FIG. 4) to 100. The SpO₂ threshold 112 remains at 85%, unchanged from FIG. 4. The alarm indicator 122 in FIG. 5 is moved over relative to the alarm indicator 122 in FIG. 4 because the SatSeconds threshold 114 is greater. In addition, two areas 126 in which the SpO₂ plot 118 drops below the SpO₂ threshold line 120 are not shaded in because the SatSeconds threshold 114 is not reached before the plot 118 again goes above the line 120. The SatSeconds symbol 124 is illustrated as a larger square in FIG. 5, corresponding to the high SatSeconds threshold 114.

FIG. 6 illustrates the difference in alarm settings when the SpO₂ threshold 112 is increased from 85% (FIG. 5) to 90% (FIG. 6). The SatSeconds threshold 114 is constant from FIG. 5 to FIG. 6. As the alarm indicator 122 and the area 126 illustrate, the SatSeconds threshold 114 is reached earlier in FIG. 6 than in FIG. 5. Because the SpO₂ plot 118 does not go above the SpO₂ threshold line 120 after the first desaturation event, calculation of the SatSeconds value is not reset. Therefore, the alarm will be activated earlier for the given plot 118.

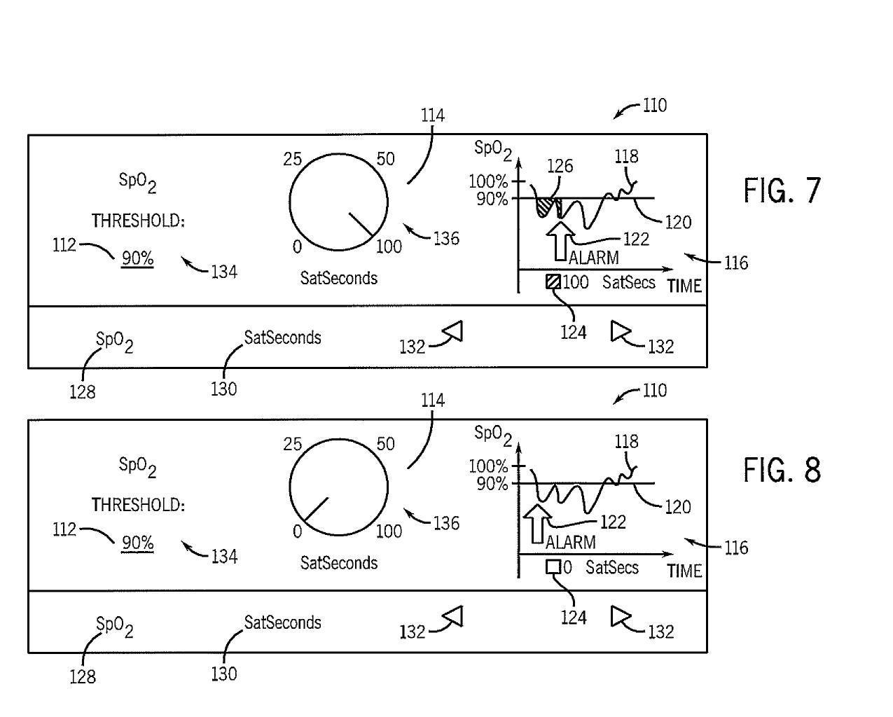

Finally, FIG. 7 illustrates the effect that reducing the SatSeconds threshold 114 to zero will have on the alarm settings. At a threshold 114 of zero, the alarm will be activated as soon as the SpO₂ plot 118 falls below the threshold line 120, as illustrated by the indicator 122. There is no shaded area 126 because the SatSeconds integration, as described above, is not needed in this example.

While only certain features have been illustrated and described herein, many modifications and changes will occur to those skilled in the art. It is, therefore, to be understood that the appended claims are intended to cover all such modifications and changes as fall within their true spirit.

What is claimed is:

1. A monitor, comprising:
a display;
a graphical user interface capable of being illustrated on the display, the graphical user interface comprising:
an indication of an alarm threshold value;
an indication of an alarm integration threshold value; and
a graphical representation of a physiological parameter, wherein the indication of the alarm threshold value generally comprises a line on the graphical representation, and the indication of the alarm integration threshold value generally comprises a shaded area on the graphical representation; and
a processor capable of calculating the physiological parameter for illustration on the display.

2. The monitor of claim 1, wherein the processor is capable of integrating the difference between the line and a real-time plot of the physiological parameter measured over time when the physiological parameter is below the line.

3 The monitor of claim 1, wherein the physical parameter comprises a blood oxygen saturation.

4. The monitor of claim 1, comprising soft keys capable of enabling user input of an alarm threshold value and/or an alarm integration threshold value.

5. The monitor of claim 4, comprising an alarm capable of alerting a caregiver when the calculated physiological parameter exceeds the alarm threshold value and/or the alarm integration threshold value.

6. The monitor of claim 1, comprising a second graphical user interface capable of being illustrated on the display, wherein the second graphical user interface comprises a real-time plot of the physiological parameter measured over time.

7. A system, comprising:
a monitor, comprising:
a graphical user interface capable of illustration on the display, the graphical user interface comprising:
an indication of an alarm threshold value;
an indication of an alarm integration threshold value; and
a graphical representation of a physiological parameter, wherein the indication of the alarm threshold value generally comprises a line on the graphical representation and the indication of the alarm integration threshold value generally comprises a shaded area on the graphical representation; and
a sensor capable of providing information to the monitor.

8. The system of claim 7, wherein the sensor comprises a pulse oximetry sensor.

9. The system of claim 7, wherein the monitor is capable of determining an alarm integration parameter based at least in part upon a real-time measurement of the physiological parameter compared to the indicated alarm threshold value line when the real-time measurement is below the line.

10. The system of claim 9, comprising an alarm capable of indicating an anomaly when an alarm integration parameter exceeds the indicated alarm integration threshold value.

11. One or more tangible, machine-readable media comprising code which, if executed by a processor, cause the processor to display a user interface, the user interface comprising:
an alarm threshold value;
an alarm integration threshold value; and
a generally graphical representation of a physiological parameter, wherein the alarm threshold value is generally illustrated as a line on the graphical representation, and the alarm integration threshold value is generally illustrated as a shaded area on the graphical representation.

12. The tangible, machine-readable media of claim 11, wherein the physiological parameter comprises a blood oxygen saturation.

13. The tangible, machine-readable media of claim 11, comprising code executable to illustrate the alarm threshold value and the line in a first color.

14. The tangible, machine-readable media of claim 11, comprising code executable to illustrate the alarm integration threshold value and the shaded area in a second color.

15. The tangible, machine-readable media of claim 11, comprising code executable to illustrate a symbol indicative of the alarm integration threshold value.

16. The tangible, machine-readable media of claim 15, comprising code executable to illustrate the alarm integration threshold value, the shaded area, and/or the symbol in a second color.
